(19) United States
(12) Patent Application Publication
(10) Pub. No.: US 2015/0191511 A1
(43) Pub. Date: Jul. 9, 2015

(54) Genetically Modified Organisms to Produce Organic Compounds

(71) Applicant: World Biotechnology LLC, North Brunswick, NJ (US)

(72) Inventors: Eugene Dinescu, North Brunswick, NJ (US); Vincent Dinescu, North Brunswick, NJ (US)

(21) Appl. No.: 14/592,071

(22) Filed: Jan. 8, 2015

Related U.S. Application Data
(60) Provisional application No. 61/925,037, filed on Jan. 8, 2014.
(60) Provisional application No. 61/929,129, filed on Jan. 20, 2014.

(51) Int. Cl. C07K 14/195; C12N 9/88

(52) U.S. Cl. CPC C07K 14/195; C12N 9/88; C12Y 401/01039

(57) ABSTRACT

Disclosed herein are embodiments for a genetically modified organism or cell line; a composition containing the same; and a method of harvesting at least one organic compound from an organism or cell line genetically engineered with an added gene for at least one carbon-fixing protein and at least one proton-pump protein.

This application claims the benefit of U.S. Provisional Application No. 61/925,037 filed Jan. 8, 2014 and U.S. Provisional Application No. 61/929,129, filed Jan. 20, 2014. The contents of these applications are hereby incorporated by reference in their entireties.

### FIELD OF INVENTION

The present disclosure is directed to creating genetically modified organisms with carbon-fixing and proton-pump proteins as well as compositions and methods to produce and harvest organic compounds, including biologics and biofuels, from the genetically modified organisms.

### BACKGROUND

Genetically modified organisms offer opportunities to more efficiently use and conserve natural resources while producing an abundance of desired organic compounds. Among these organic compounds are biologics and biofuel. To increase production efficiency of a biologic or biofuel, either the quantity of input must be reduced, the rate of production must be increased or the quality of the product must be improved. These areas can be addressed by boosting efficiency through providing an energy source from broad spectrum light and utilizing alternative carbon sources, such as carbon dioxide and methane.

To create cellular products, such as biologics and bioethanol, organisms require an energy source in the form of adenosine triphosphate (ATP). In order to generate ATP, many organisms must find and consume food from outside sources. These food sources, however, are generally organic carbon sources such as plants or other organisms. The consumption of food as an energy source uses organic carbon sources that may become increasingly scarce and may be better utilized in other ways.

In contrast, some organisms, such as plants and algae, are able to create food sources from alternative carbon sources (i.e., carbon dioxide, CO₂, or methane, CH₄) through process like photosynthesis or chemosynthesis. These organisms are able to convert carbon dioxide or methane into a usable from of energy such as sugars, starches or biofuels. Several pathways are known to use carbon dioxide to create a range of carbohydrate products; these pathways include the Calvin Cycle, C₄ carbon fixation and Crassulacean acid metabolism (CAM) photosynthesis, among others. Unfortunately, relatively few organisms are able to perform these pathways which remain inefficient in creating energy sources. Thus, there exists a need in the art for a system to increase the utilization of alternative carbon sources while minimizing the input of resources and energy.

In addition to using food sources, many organisms also use a proton gradient to create ATP. Generally, ATP is produced when a proton gradient is created across a cellular membrane. This proton motive force drives the production of ATP as protons move down the gradient through an ATP synthase. While this proton gradient can ultimately generate energy in the form of ATP, there is also an ATP energy cost to first pump protons across the membrane and against the gradient. There exists a need in the art for a system to minimize the energy costs when creating this proton gradient and to increase ATP output while avoiding cellular pathways that have negative feedback loops.

Disclosed herein is a genetically modified organism or cell line as well as a method and composition which utilizes genetically modified organisms and cell lines to use carbon-fixing and proton-pump proteins to increase ATP production and improve the organism's or cell line's productivity. Carbon-fixing proteins, such as ribulose-1,5-bisphosphate carboxylase/oxygenase (RuBisCO), are able to create energy sources using otherwise underutilized resources like inorganic carbon from carbon dioxide. Certain proton-pumps, such as bacteriorhodopsin, are light-driven and can create a proton gradient utilizing the light absorbed by the sun or other light source as an energy source. By relying on carbon-fixation and sunlight for energy, the organism or cell line does not require as much energy from other energy sources, such as glucose, glycogen, trehalose, NADH and FADH₂, to produce ATP. As a result, the organisms can be more efficient in their energy usage and production of other compounds.

### OBJECTS AND SUMMARY OF THE INVENTION

It is an object of certain embodiments of the present invention to provide an organism or cell line genetically modified with an added gene for at least one carbon-fixing protein and at least one proton-pump protein to help to produce organic compounds, such as biologics and biofuels.

It is an object of certain embodiments of the present invention to provide genetically modified bacteria, fungi, plants, fish, birds, mammals or cell lines to include at least one carbon-fixing protein and at least one light-driven, proton-pump protein.

It is an object of certain embodiments of the present invention to provide an organism or cell line genetically modified with a carbon-fixing protein and a proton-pump protein to include a membrane targeting sequence.

It is an object of certain embodiments of the present invention to provide an organism or cell line genetically modified with an added proton-pump protein including a mitochondrial targeting sequence so that the proton-pump protein is inserted into the inner mitochondrial membrane.

It is an object of certain embodiments of the present invention to provide an organism or cell line genetically modified to increase the titer of a biologic or biofuel.

It is an object of certain embodiments of the present invention to provide an organism or cell line genetically modified to utilize alternative carbon sources as an energy resource.

It is an object of certain embodiments of the present invention to provide an organism or cell line genetically modified to increase production of a biologic where the biologic may be allergenics, antibodies, blood products or derivatives thereof, enzymes, growth factors, hormones, immunomodulators, interferons, interleukins, polypeptides, proteins, serums, tissues, toxins or vaccines.

It is an object of certain embodiments of the present invention to provide an organism or cell line genetically modified to increase production of a biofuel where the biofuel may be biodiesel, biogas, butanol, ethanol or methanol.

It is an object of certain embodiments of the present invention to provide an organism or cell line genetically modified to increase the production of ATP and organic compounds without increasing production input.

It is an object of certain embodiments of the present invention to provide an organism or cell line genetically modified to increase ATP-dependent cellular functions.

It is an object of certain embodiments of the present invention to provide an organism or cell line genetically modified to require fewer resources for growth and organic compound production.

### BRIEF DESCRIPTION OF THE FIGURES

FIG. 1 is a generalized flow chart demonstrating the process for selecting an organism or cell line and integrating selected genes to be used in harvesting organic compounds.

### DETAILED DESCRIPTION

The present invention is directed to a genetically modified organism or cell line genetically engineered with an added gene for at least one carbon-fixing protein and at least one proton-pump protein; a composition with an organism or cell line genetically engineered with a gene for at least one carbon-fixing protein and at least one proton-pump protein; and a method of producing organic compounds from an organism or cell line genetically engineered with an added gene for at least one carbon-fixing protein and at least one proton-pump protein. In particular embodiments, the organic compound is a biologic or a biofuel.

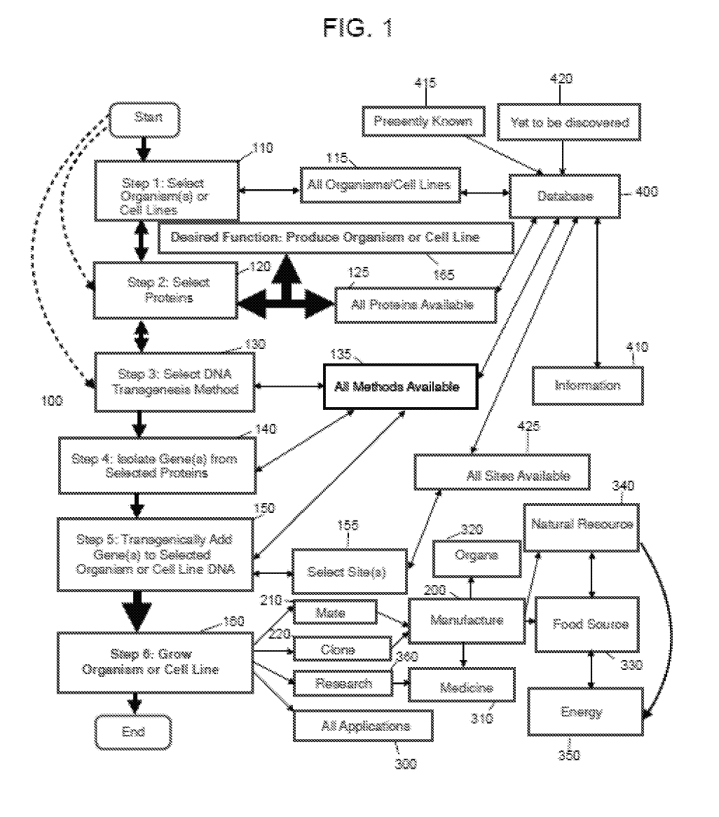

FIG. 1 shows a flow chart demonstrating the disclosed invention. In some embodiments, the invention may create the genetically engineered organism or cell line and may include a genetic engineering function process 100, organism or cell line manufacturing 200, a plurality of additional applications 300 and databasing 400. The genetic engineering function process 100 may include a selection of at least one organism or cell line 110, a protein selection of at least one carbon-fixing protein and at least one proton-pump protein 120, a transgenic methodology 130, gene isolations 140, gene additions 150 and organism or cell line growth 160.

The organism or cell line growth 160 may result in a genetically modified organism or cell line 165. The genetically modified organism or cell line may then develop as usual. The genetically modified organism or cell line may then express/produce at least one carbon-fixing protein and at least one proton-pump protein.

The organism selection 110 may include selecting from any known organism or cell lines 115. Selection may include all presently known organisms or cell lines 415 or all yet to be discovered or created organisms or cell lines 420.

The protein selection 120 may include all available proteins 125 including all carbon-fixing proteins and all proton-pump proteins. In some embodiments, the carbon-fixing protein may include those proteins involved in any carbon-fixing pathway. The carbon-fixing proteins may enable the organism or cell line to use alternative carbon sources, such as carbon dioxide or methane, to make carbohydrates and other energy sources. The protein selection 120 may also include additional proteins involved in photosynthesis or chemosynthesis.

The protein selection 120 may include selecting RuBisCO, an enzyme involved in the first step of carbon fixation, the process by which atmospheric carbon dioxide is converted to energy-rich molecules such as carbohydrates, sugars, starches as well as a variety of other molecules. During carbon fixing, RuBisCO attaches an atmospheric carbon dioxide molecule to ribulose 1,5-bisphosphate. The product of this reaction is an unstable six-carbon phosphorylated intermediate called 3-keto-2-carboxyarabinitol-1,5-bisphosphate, that will decay almost immediately into two molecules of glycerate-3-phosphate. The glycerate-3-phosphate can be used to produce larger molecules for the organism, including a variety of compounds as energy sources. Other chemosynthetic proteins may be used which can utilize carbon dioxide, methane and hydrogen sulfide to create large carbohydrates and other compounds.

The protein selection 120 may also include selecting bacteriorhodopsin, an integral membrane protein that has Vitamin A attached. Bacteriorhodopsin effectively absorbs green light (wavelength 500-650 nm, with absorption maximum obtained at 568 nm) and is a protein used by archaea organisms. The bacteriorhodopsin is a proton-pump which changes conformation once it absorbs light and pumps protons through a membrane. The bacteriorhodopsin may be expressed in mitochondria and may allow ATP production via light absorption as chemical energy.

In certain embodiments, protein selection 120 may include proton-pump proteins which include those proteins with conformational changes upon light absorption and pump protons through a membrane. The proton-pump protein may allow the organism to produce energy via ATP synthase. The proton-pump protein may be expressed in mitochondria and may allow ATP production via light absorption. In certain embodiments, the proton-pump protein may be a combination of two or more proteins that can be utilized to enhance or facilitate the light absorption function.

The transgenic methodology 130 may include all methods known to those of skill in the art 135 to perform transgenesis.

The gene isolation 140 may include isolating the genes that codes for a carbon-fixing protein and a proton-pump protein by any known method to those of skill in the art. The gene isolation 140 may include using restriction enzymes and gel electrophoresis. In certain embodiments, polymerase chain reaction (PCR) may be used to amplify the gene segment. In an alternate embodiment, the gene sequence for the carbon-fixing and proton-pump proteins may be found in known DNA databases 400.

Gene addition 150 may include transgenically adding the genes to the genome of the selected organism or cell line 110 by all methods available 135. In certain embodiments, the gene addition 150 occurs at a selected site in the organism's or cell line's genome 155 which may include all known sites available 425. In other embodiments, the invention may add the gene to the germ line of the selected organism 110, by any known method 150 to those of skill in the art, including, but not limited to, by injecting the foreign DNA into the nucleus of a fertilized ovum.

It should be understood that the organism or cell line selection 110, the protein selection 120 and the transgenic methodology 130 may be performed in any combination and does not need to follow any particular order.

The genetically modified organism or cell line manufacturing 200 may include mating 210, cloning 220 as well as other known methods to those of skill in the art. The genetically modified organism or cell line manufacturing 200 may also include engineering for desired characteristics through any known method to those of skill in the art, such as cross-breeding or the like. For example, if enzyme A, B, C, D, E, and F are needed for complete amino acid synthesis in organism AA one can transgenically create a total of 6 organisms that all express a single missing enzyme. One organism will express A, another B, another C, another D, another E, and another F. Once this is accomplished these organisms can be crossbreed until production of a hybrid that expresses each enzyme is attained.

The plurality of additional applications 300 may include research 360, medicine 310, stem cell research and host organism production 320, a production of food source 330 or natural resource 340 including energy production 350.

The databasing 400 may include producing a database of all possible types of combinations and information 410 for producing the desired organism or cell line 165.

While FIG. 1 shows a flow chart demonstrating versions of the invention, it should be understood the invention may be performed in any combination and does not necessarily need to be in any order.

### Carbon-Fixation Technology

The current invention may utilize a carbon-fixing protein to help utilize the use of alternative carbon sources, such as carbon dioxide or methane.

Many organisms, including cyanobacteria, algae and plants, use photosynthesis and chemosynthesis to create energy and other resources. Generally, photosynthesis is the process in which atmospheric carbon dioxide is incorporated into a variety of organic compounds with solar energy. Similarly, chemosynthesis use carbon sources, generally either carbon dioxide or methane, to make organic compounds with hydrogen gas or hydrogen sulfide as energy sources.

In some embodiments of the present invention, the genetically modified organism or cell line may include at least one carbon-fixing protein involved in photosynthesis or chemosynthesis. In other embodiments, the carbon-fixing protein may be used in the Calvin-Benson-Bassham Cycle, C₄ Cycle, Crassulacean acid metabolism (CAM), Wood-Ljungdahl (Reductive Acetyl Coenzyme A (Acetyl-CoA)) Pathway, Fuchs-Holo (3-hydroxypropionate (3-HP)) Bi-cycle, 3-hydroxypropionate/4-hydroxybutyrate (3-HP/4-HB) Cycle, dicarboxylate/4-hydroxybutyrate (DC/4-HB) Cycle or Arnon-Buchanan (Reductive Tricarboxylic Acid (rTCA)) Cycle.

In particular embodiments, the carbon-fixing protein may be selected from, but not limited to, ribulose 1,5-bisphosphate carboxylase, phosphoenolpyruvate (PEP) carboxylase, 2-oxoglutarate synthase, carbon monoxide dehydrogenase, carbon monoxide-methylating acetyl-CoA synthase, carbon monoxide-acetyl-CoA synthase, acetyl-CoA carboxylase, propionyl-CoA carboxylase, biotin-dependent acetyl-CoA/propionyl-CoA carboxylase, formylmethanofuran dehydrogenase, pyruvate synthase, pyruvate oxidoreductases, pyruvate:ferredoxin oxidoreductase, 2-oxoglutarate:ferredoxin oxidoreductase, isocitrate dehydrogenase, formate dehydrogenase, NADP-dependent formate dehydrogenase and α-ketoglutarate:ferredoxin oxidoreductase. In some embodiments, the genetically modified organism or cell line further comprises at least one additional carbon-fixing protein.

In some embodiments, the proton-pump protein and the carbon-fixing protein are in different cellular compartments. In other embodiments, the proton-pump protein and the carbon-fixing protein are in the same cellular compartments. In still other embodiments, the carbon-fixing protein is located in the cytoplasm, mitochondria or cellular membranes.

In other embodiments, the genetically modified organism or cell line further comprises at least one additional protein to enhance or facilitate the carbon-fixing protein. In some particular embodiments, the carbon-fixing protein is genetically engineered to have a higher affinity for carbon dioxide.

In other embodiments, the genetically modified organism or cell line further comprises at least one additional photosynthetic or chemosynthetic protein. The additional photosynthetic or chemosynthetic protein, may include, but is not limited to, RuBisCO activase, phosphoglycerate kinase, triose phosphate isomerase, aldolase, fructose-1,6-bisphosphate phosphatase, transketolase, sedoheptulose-1,7-bisphosphate phosphatase, ribulose-5-phosphate epimerase, ribose-5-phosphate isomerase, glyceraldehyde 3-phosphate dehydrogenase, sedoheptulose-1,7-bisphophatase, malate dehydrogenase, aspartate aminotransferase, NADP:malic enzyme, phosphoenolpyruvate carboxykinase (PEPCK), pyruvate-phosphate dikinase, carbonic anhydrase, phosphoribulokinase (PRK), ATP-citrate lyase, malonyl-CoA reductase, propionyl-CoA synthase, malyl-CoA lyase, methylmalonyl-CoA mutase, 4-hydroxybutyryl-CoA dehydratase, methenyl-tetrahydromethanopterin cyclohydrolase, methylene-tetrahydromethanopterin dehydrogenase, methylene-tetrahydromethanopterin reductase, carbon monoxide dehydrogenase-acetyl-CoA-synthase, succinyl-CoA synthetase, fumarate reductase, fumarate hydratase, phosphoenolpyruvate synthase, acetoacetyl-CoA β-ketothiolase, (S)-3-hydroxybutyryl-CoA dehydrogenase, crotonyl-CoA dehydratase, 4-hydroxybutyrate-CoA ligase, succinic semialdehyde reductase, succinyl-CoA reductase, methylmalonyl-CoA epimerase, acryloyl-CoA reductase, 3-hydroxypropionyl-CoA dehydratase, 3-hydroxypropionyl-CoA ligase, malonic semialdehyde reductase, methyl transferase, methyl-accepting corrinoid protein, ATP sulfurylase, methane monooxygenase, methanol dehydrogenase, NiFe hydrogenases, carcinoid-iron sulfur protein, rhodanese and APSreductase.

In other embodiments, the genetically modified organism or cell line further comprises coenzymes. The coenzyme may include, but is not limited to, tetrahydropterin, acetyl coenzyme A (acetyl-CoA), B₁₂, cobalamin, nicotinamide adenine dinucleotide (NAD), flavin adenine dinucleotide (FAD) and biotin.

In some embodiments, the genetically modified organism or cell line further comprises adjusted hormone levels. In other embodiments, the adjusted hormone levels increase the rate of growth and organic compound production in the organism or cell line as compared to a wild type organism or cell line. In other embodiments, the genetically modified organism or cell line further comprises adjusted promoter responsibility.

In particular embodiments, the organism or cell line may produce an increased amount of biologic, biofuel, fatty acids or food sources as compared to a wild type organism or cell line; also, the genetically modified organism or cell line may require less food resources. In other embodiments, genetically modified organism or cell line may produce a pH-adjusting soil additive.

### Direct-Light Technology

The current invention may utilize proton-pump protein to help create a proton gradient across a cell membrane, including a light-driven, proton-pump protein. When a light-driven, proton-pump protein absorbs light, the protein generally forms a channel through a cellular membrane and undergoes a series of conformational changes in response to the absorbed light. The conformational changes allow protons to pass to and from different amino acid groups along the protein channel and through a cellular membrane. Moving the protons through the proton-pumps allows a proton gradient to be formed with enough proton motive force to drive an ATP synthase to make ATP. In some embodiments of the current invention, the light-driven, proton-pump protein may be archaerhodopsin, bacteriorhodopsin, opsin, proteorhodopsin, rhodopsin, xanthorhodopsin, homologs or combinations thereof. In certain embodiments, the proton-pump protein may include bacteriorhodopsin, an integral membrane protein that has Vitamin A attached and ability to absorb light.

Proton-pump proteins are able to operate after absorbing light from a wide range of the light spectrum. For instance, bacteriorhodpsin may generally best absorb light between 500 nm to 650 nm which corresponds to green light in the visible spectrum. Likewise, proteorhdopsin may maximally absorb light around 525 nm (green light) as well as around 490 nm (blue light) and rhodopsin will generally absorb light from around 490 nm to around 510 nm. While these are optimal ranges, proton-pump proteins are able to absorb light well above and below these peaks. In other embodiments, the proton-pump protein may absorb light between about 100 nm and about 1 μm, between about 300 nm and about 750 nm, between about 450 nm and about 650 nm, between about 450 nm and about 550 nm and between about 550 nm and about 600 nm.

In some embodiments of the present invention, the genetically modified organism or cell line is able to produce a variety of organic compounds, including biologics or biofuels. Biologics are generally considered to be large, complex molecules which are often produced by living cells and organisms naturally or through genetic engineering. Biologics may be used for a variety of uses, including disease treatments, diagnostics and prevention of a variety of health conditions. Unlike drugs, which can be produced on a large scale by chemical means, it remains very difficult to reproduce biologics outside of a living organism or cell line. To help solve this problem, embodiments of the current invention will utilize living organisms and cell lines to increase production of biologics. In some embodiments, the biologic may be allergenics, antibodies, blood products or derivatives thereof, enzymes, growth factors, hormones, immunomodulators, interferons, interleukins, polypeptides, proteins, serums, tissues, toxins and vaccines.

In particular embodiments, where the biologic is an antibody, the antibody may be, but not limited to, antitoxins, IgA, IgD, IgE, IgG, IgM antibodies or combinations thereof and may be either a monoclonal, polyclonal or bispecific antibody. In other embodiments, where the biologic is a blood product, the blood product may be, but not limited to, red blood cells, blood plasma, white blood cells, platelets, derivatives thereof or combinations thereof.

In some embodiments, where the biologic is an enzyme, the enzyme may be, but not limited to, an amidase, amylase, catalase, cellulase, dehydrogenase, endonuclease, hemicellulase, hydrolase, isomerase, kinase, ligase, lipase, lyase, lysozyme, pectinase, peroxidase, phosphatese, polymerase, protease, oxidase, oxidoreductase, reductase, transferase or combinations thereof.

In other embodiments, where the biologic is a hormone, the hormone may be, but is not limited to, adiponectin, adrenocorticotropic hormone, androgen, angiotensinogen, antidiuretic hormone, amylin, atrial-natriuretic peptide, brain natriuretic peptide, cacitonin, cholecystokinin, cortisol, corticotrophin-releasing hormone, cortistatin, enkephalin, endothelin, epinephrine, estrogen, erythropoietin, follicle-stimulating hormone, galanin, gastric inhibitory polypeptide, gastrin, ghrelin, glucagon, glucagon-like peptide-1, glucocorticoid, gonadotropin-releasing hormone, growth hormone, growth hormone-releasing hormone, hepcidin, human chorionic gonadotropin, human placental lactogen, humoral factors, inhibin, insulin, insulin-like growth factor, leptin, leukotriene, lipotropin, luteinizing hormone, melatonin, melanocyte stimulating hormone, mineralocorticoid, motilin, orexin, oxytocin, pancreatic polypeptide, parathyroid, pituitary adenlate cyclase-activating peptide, progesterone, prolactin, prolactin releasing hormone, prostacyclin, prostaglandins, relaxin, renin, secosteroid, secretin, somatostatin, testosterone, thrombopoietin, thromboxane, thyroid-stimulating hormone, thyrotropin-releasing hormone, thyroxine, triiodothyronine, vasoactive intestinal peptide or combinations thereof.

In addition, in certain embodiments, a genetically modified organism or cell line may have additional genes for certain hormones included to increase hormone production within the organism or cell line. This may provide an additional production advantage to the genetically modified organism or cell line. Additionally, the genetically modified organism or cell line may be injected with peptide hormones responsible for hormone production.

In still other embodiments, where the biologic is an interferon, the interferon may be, but not limited to, interferon type I, interferon type II, interferon type III or combinations thereof. In some embodiments, where the biologic is an interleukin, the interleukin may be, but not limited to, interleukin-1, interleukin-2, interleukin-3, interleukin-4, interleukin-5, interleukin-6, interleukin-7, interleukin-8, interleukin-9, interleukin-10, interleukin-11, interleukin-12, interleukin-13, interleukin-14, interleukin-15, interleukin-16, interleukin-17, interleukin-18, interleukin-19, interleukin-20, interleukin-21, interleukin-22, interleukin-23, interleukin-24, interleukin-25, interleukin-26, interleukin-27, interleukin-28, interleukin-29, interleukin-30, interleukin-31, interleukin-32, interleukin-33, interleukin-34, interleukin-35, interleukin-36, interleukin-37 or combinations thereof.

In particular embodiments, where the biologic is a vaccine, the vaccine may be, but not limited to a whole-cell vaccine, DNA vaccine, RNA vaccine, protein-based vaccine, peptide-based vaccine, attenuated organism vaccine, attenuated virus or combinations thereof. For this particular embodiment, the vaccine may, but is not limited to, providing immunity from African swine fever, anthrax, bubonic plague, cervical cancer, chicken pox, Coxsackie, dengue fever, diphtheria, Ebola, echovirus, encephalitis, gastroenteritis, hepatitis, herpes, human immunodeficiency disease (HIV-1 or HIV-2), influenza, lower respiratory tract infection, Lyme disease, Marburg, measles, monkeypox, mumps, Norwalk virus infection, papillomavirus, parainfluenza, parvovirus, pertussis, picorna virus infection, pneumonia, pneumonic plague, polio, rabies, rotavirus infection, rubella, shingles, smallpox, swine flu, tetanus, tuberculosis, typhoids or yellow fever.

In addition, some embodiments of the present invention may be used to treat conditions including, but not limited to, ankylosing spondylitis, autoimmune diseases, cancer, Crohn's disease, diabetes, gout, indeterminate colitis, inflammatory bowel disease, psoriasis, psoriatic arthritis, rheumatoid arthritis, ulcerative colitis, uveitis and viral infection.

Embodiments of the current invention may also be used to increase production of biofuels. Biofuels are considered to be energy sources derived from organic materials. Biofuels are most widely used in liquid form which may be more easily integrated into currently used systems; ethanol as a biofuel is particularly known for this feature. Biofuels also have the feature of being transportable sources of energy. The use of biofuels may be preferable to other renewable energy sources, such as wind, solar, hydrothermal and tidal flows, which would require additional input to make these other energy sources compatible with presently used infrastructure. Biofuels may be produced through fermentation of organic material or through extraction of lipids, vegetable oils and animal fats.

To increase the efficiency of production of ethanol, some embodiments of the current invention allows genetically modified organisms and cell lines to increase the amount of ethanol and other biofuels produced during manufacturing. Embodiments of the current invention may increase the carbon compounds, proton gradient and available ATP which will then provide the modified organism or cell line with an abundance of energy to increase production of a biofuel. In some embodiments, where the organic compound is a biofuel, the biofuel may be, but is not limited, biodiesel, biogas, butanol, ethanol or methanol. In particular embodiments, the biofuel may be ethanol. In certain other embodiments, the invention may be used for other biomolecules including, but not limited to, spider silk, cartilage, exoskeleton structures and the like. In particular embodiments, the invention may be used to create lighter and stronger machines, like vehicles, that may be capable of regeneration through carbon-fixing.

Typically, proton-pumping proteins are located in a cellular membrane. In order to effectively create a proton gradient, protons need to be kept separated-usually by the cellular membrane. In prokaryotic cells (i.e., cells without membrane-bound organelles, including mitochondria), proton-pump proteins may be located in plasma among other membranes. In some embodiments, the proton-pump protein, as well as the carbon-fixing protein, may be integrated into the inner mitochondrial membrane. In particular embodiments, the carbon-fixing protein and the proton-pump protein may be integrated into two or more cellular membranes. In some embodiments, the carbon-fixing protein and the proton-pump protein may be integrated into the same cellular membranes and, in other embodiments, the carbon-fixing protein and the proton-pump proteins may be integrated into different cellular membranes. Likewise, for other embodiments, two different carbon-fixing proteins or proton-pump proteins may be integrated into the same or different cellular membranes and may be integrated into two or more cellular membranes.

One feature of the current invention is the creation and maintenance of a proton gradient. In order to create this gradient, the proton-pump protein should be integrated into at least one cellular membrane and correctly oriented in relation to the native ATP synthase. To ensure a protein is properly placed into a membrane, a protein gene may have a membrane targeting sequence. After a protein is translated, the targeting sequences enable the cellular machinery to transport the protein to its proper location. In other embodiments, the gene for the proton-pump protein may be genetically engineered to include at least one membrane targeting sequence. In particular embodiments, the targeting sequence may be a mitochondrial targeting sequence or other specific membrane targeting sequence. Where some of the embodiments use a mitochondrial targeting sequence, the mitochondrial targeting sequence may be from the ATP, COX IV or RIP1 genes or homologs thereof.

Likewise, a carbon-fixing protein may also be genetically engineered in some embodiments to include a membrane targeting sequence, mitochondrial targeting sequence (including the mitochondrial targeting sequence may be from the ATP, COX IV or RIP1 genes or homologs thereof) or other specific membrane targeting sequence.

### Invention Systems and Technologies

In some embodiments, the invention may include genetically engineered genes. In particular embodiments, the gene of the carbon-fixing protein and of the proton-pump protein are genetically engineered to include a selectable marker. To ensure a gene is properly integrated into the genome of the targeted organism or cell line, a selectable marker may be used. A selectable marker is generally a gene or part of a gene which is also inserted with a gene of interest. The selectable marker provides an additional, non-native characteristic to the organism or cell line to distinguish the organisms or cell lines with the gene of interest and selectable marker from the organisms and cell lines without it.

In some embodiments, the selectable marker may be a drug resistance marker, a multidrug resistance marker, a metabolic survival marker, a color marker, a fluorescent marker or a combination thereof. In particular embodiments, the selectable marker may be dihydrofolate reductase gene, a guanosine phosphoribosyl transferase (GPT) gene, histidinol resistance gene, hygromycin resistance gene, β-galactosidase gene, green fluorescent protein gene, red fluorescent protein gene, blue fluorescent protein gene, yellow fluorescent protein gene, dsRed fluorescent protein gene, zeomycin resistance gene, zeocin resistance gene, puromycin resistance gene, Blacsticidin S resistance gene, spectinomycin resistance gene, streptomycin resistance gene and a neomycin resistance gene.

The invention may require integration of the carbon-fixing, proton-pump or other genetically engineered genes that are not native to a selected organism's or cell line's genome. To integrate the carbon-fixing and proton-pump genes into a genome, a variety of techniques, known to those skilled in the art, may be used. Some embodiments may use genetically engineering an organism or cell with techniques including, but not limited to, breeding, calcium phosphate precipitation, chemical poration, cloning, conjugation, DEAE-dextran mediated transfection, electroporation, homologous recombination, non-homologous recombination, laser irradiation, lipofection, natural transformation, magnetofection, microinjection, particle bombardment, PEG poration, protoplast fusion, retroviral delivery, silicon fiber delivery, sonoporation, transfection, transformation or transduction. In particular embodiments where genetic engineering is through transduction, a lentivirus may be used.

Some organisms, such as with Saccharomyces cerevisiae or Schizosaccharomyces pombe, are unable to produce retinal which is a required co-factor for the proper functioning of bacteriorhodopsin. In some embodiments, depending on the organism or cell line used, the invention further comprises growing the organism or cell line with co-factor retinal. As an alternative in other embodiments, an organism or cell line which is unable to produce retinal may be genetically engineered to include genes for crtE, crtYB, crtI and Bcmo1 or homologs thereof which may be used as a set of genes to make β-carotene which may be converted to retinal. Likewise, in some embodiments, the invention may further comprise growing the genetically modified organism or cell line with coenzymes, such as NAD and FAD. In other embodiments, the genetically modified organism or cell line may be further engineered to include the genes involved in the biosynthesis of coenzymes.

A variety of organisms or cell lines may be used in the invention. The organism may be selected from the bacterial, eukaryotic and archaic kingdoms, which may include, but not limited to, bacteria, fungi, plants, fish, birds and mammals.

In some embodiments, the organism may be a bacterium. The bacteria may be, but not limited to, Agrobacterium tumefaciens, Bacillus brevis, Bacillus licheniformis, Bacillus subtilis, Cyanobacteria, Escherichia coli, Paenibacillus, Penicillium griseofulvum, Pseudomonas fluorescens, Ralstonia eutropha, Streptomyces aureofaciens, Streptomyces fradiae, Streptomyces lincolnensis, Streptomyces rimosus or Streptomyces venezuelae.

In other embodiments, the organism may be a fungus. In particular cases, the fungus may be a yeast. The fungus may be, but not limited to, Acremonium chrysogenum, Aspergillus awamori, Aspergillus nidulans, Aspergillus niger, Aspergillus rugulosus, Chrysosporium lucknowense, Hansenula polymorpha, Pichia pastoris, Saccharomyces cerevisiae or Schizosaccharomyces pombe.

In other embodiments, the organism may be an archaea. The archaea may be, but not limited to, Halobacterium salinarum or Pyrolobus fumarii.

In further embodiments, the organism may be a plant. The plant may be, but not limited to, alfalfa, algae, Arabidopsis thaliana, banana, bean, beet, Camelina sativa, canola, carrot, corn, legumes, palm, potato, rapeseed, rice, safflower, soybean, spinach, strawberry, sugarcane, sunflower, tobacco, tomato, turnip or wheat.

In particular embodiments, the plant may be an algae. The algae may be, but not limited to, Ahnfeltia, Alaria esculenta, Ankistrodesmus, Ascophyllum nodosum, Betaphycus gelatinum, Botryococcus braunii, Callophyllis variegate, Caulerpa, Chlorella protothecoides, Chlorella vulgaris, Chlamydomonas reinhardtii, Chondrus crispus, Cladosiphon okamuranus, Crypthecodinium cohnii, Dunaliella bardowil, Dunaliella salina, Dunaliella tertiolecta, Durvillaea, Ecklonia, Eucheuma, Gelidiella acerosa, Gelidium, Gracilaria, Haematococcus pluvialis, Hantzschia, Hizikia fusiformis, Isochrysis galbana, Kappaphycus, Laminaria, Lessonia, Macrocystis pyrifera, Mastocarpus stellatus, Monostroma, Mazzaella, Nannochloris, Nannochloropsis, Neochloris oleoabundans, Nitzschia, Palmaria palmate, Phaeodactylum tricornutum, Phymatolithon, Pleurochrysis carterae, Porphyra, Porphyridium, Sarcothalia, Sargassum, Scenedesmus, Schiochytrium, Stichococcus, Tetraselmis suecica, Thalassiosira pseudonana, Ulva or Undaria. In still other embodiments, the algae may be Botryococcus braunii, Isochrysis galbana or Neochloris oleoabundans. In particular embodiments, the algae may be genetically engineered to have a higher lipid content in comparison to non-genetically modified algae.

In some embodiments, the organism may be a fish. The fish may be, but not limited to, carp, catfish, goldfish, loach, medaka, salmon, tilapia, trout or zebra fish.

In some embodiments, the organism may be a bird. The bird may be, but not limited to, a blackbird, canary, chicken, cockatoo, crow, duck, eagle, emu, falcon, finch, goose, hawk, jay bird, kiwi, macaw, mynah, ostrich, parakeet, parrot, partridge, pigeon, pheasant, quail, rhea, sparrow, toucan, turkey or warbler.

In other embodiments, the organism may be a mammal. The mammal may be, but not limited to, a bison, buffalo, bull, camel, cow, donkey, goat, horse, llama, mouse, non-human primate, oxen, pig, rabbit, rat or sheep.

In some embodiments, the invention may utilize a cell line. In particular embodiments, the cell line may be a suspension cell line. In other embodiments, the cell line may be, but not limited to, 3T3, A549, Be2C, Caco2, CHO, Cos7, GT293, HEK 293, HepG2, HL60, HT1080, hybridoma, IMR90, Jurkat, K562, LnCap, MCF7, myeloma, N50, Namalwa, PC12, PER.C6, primary fibroblast, SKBR3, SW480, THP1, U 266B1, U937, WEHI 231 and YAC 1. In still other embodiments, the cell line may be A549, HeLa, HEK 293, Jurkat or 3T3.

### Functions of the Invention

The genetically modified organism or cell line may be used to help increase the productivity of a variety of cellular functions. Increasing these cellular functions may provide a greater yield of organic products, including for biologics and biofuels. In some embodiments, the genetically modified organism or cell lines may increase ATP production. As previously mentioned, ATP is produced, in part, from the breakdown of carbohydrates or from a proton travels through an ATP synthase which provides sufficient energy to bind a phosphate group to adenosine diphosphate creating ATP. By incorporating a greater number of carbon-fixing and proton-pump proteins, more energy sources will be available to generate ATP. Thus, some embodiments may increase availability of carbohydrate sources and the proton gradient. Also, other embodiments may also increase ATP synthase activity as well. In some embodiments, high titers of organic compounds, including biologics and biofuels, may be produced from the increase of available energy.

In some embodiments, ATP-dependent cellular functions may also increase. Many cellular functions required energy in the form of ATP in order to occur. These cellular functions include metabolic reactions, macromolecule syntheses (i.e., DNA, RNA, proteins, carbohydrates, amino acids, lipids, fatty acids, ethanol etc.), signaling, fermentation, cell structure, cell movement, mitosis, meiosis, among many others. Also, by providing a possible alternative source of ATP, in other embodiments, the addition of the carbon-fixing and proton-pump proteins may help to increase cellular energy conservation.

The possible increased amount of ATP may help increase protein folding. For example, when a protein is targeted to the mitochondrial membrane (such as a proton-pump protein with a mitochondrial targeting sequence), the protein must be unfolded from its native state in order to be imported into the mitochondria. Another example is when a protein is overexpressed, it may misfold and form an aggregate or it may be degraded. For these proteins to be correctly refolded, the process requires energy in the form of ATP along with the energy needs of chaperone, transport and other necessary proteins. In some embodiments, the additional carbon-fixing and proton-pump proteins may increase the supply of ATP to increase protein folding.

In some embodiments, the genetically modified organism or cell line may also help to increase production of various cellular products including, but not limited to, fatty acids, amino acids and ethanol and their synthesis. Fatty acids are important sources for energy and cellular structures as well as being utilized for biofuel production. However, fatty acids require ATP in their synthesis. Fatty acid synthesis occurs through the Type-I and Type-II fatty acid synthases and is encoded by the FASN gene and its homologs thereof. In some embodiments, organisms or cell lines may be further genetically engineered to also include the FAS1, FAS2, FASN genes, homologs or combinations thereof. The addition of the FAS1, FAS2, FASN genes, homologs or combinations thereof may help to increase fatty acid synthesis in some embodiments. Likewise, an increase in fatty acid production may increase the amount of animal fat in genetically modified organisms and cell lines which may increase the amount of biofuel and biodiesel produced.

Amino acids are essential for protein synthesis which also requires ATP energy input. Production of amino acids varies greatly depending on the organism. In some organisms, particular amino acids cannot be synthesized by the organism, as seen with humans. In some embodiments, organisms or cell lines may be further genetically engineered to also include the gene for an amino acid producing enzyme. In particular embodiments, the amino acid producing enzyme may be for the production of an amino acid including, but not limited, alanine, arginine, aspartate, asparagine, cysteine, glutamate, glutamine, glycine, histidine, isoleucine, leucine, lysine, methionine, phenylalanine, proline, serine, threonine, tyrosine, tryptophan or valine. In particular embodiments, the gene for the enzyme for amino acid synthesis may before the synthesis of lysine from aspartate.

In certain embodiments, the increased production of hormones, amino acids and fatty acids of the genetically modified organisms or cell line may help the genetically modified organism or cell lines to decrease dietary intake, grow faster than their non-genetically modified counterparts and be mass-produced for less than the current cost of production.

During normal respiration, organisms are able to use oxygen to convert sugars and carbohydrates into energy in the form of ATP. During fermentation, however, oxygen is not available and organisms (generally yeast and some bacteria) utilize the alternative fermentation process. The result of this process is continual production of ATP with alcohol, such as ethanol, being created as a byproduct. In some embodiments, the genetically modified organism or cell line may increase ethanol production by providing a continuing proton gradient and additional carbon energy sources.

In embodiments of the invention, the production of a proton gradient outside of the normal cellular processes may increase the production or synthesis of fatty acids, amino acids and ethanol.

The additional ATP may also help organisms and cell lines counteract the stresses associated with ethanol production. When ethanol levels reach a critical level within the cell, the ethanol can begin to denature proteins and increase membrane fluidity. If a membrane becomes too fluid, the proton gradient may be lost and other energy sources, such as glucose, glycogen, trehalose, NADH and FADH₂, may be consumed at greater rates to preserve the proton gradient. Denatured proteins may also begin to aggregate preventing the protein from properly functioning or trigger other cellular mechanisms to degrade and recycle those proteins.

In some embodiments, the genetically modified organism or cell line may have increased ethanol tolerance. Cellular defenses against ethanol stresses include the upregulation of fatty acid elongation factor Elo1. Elo1 increases the proportion of acyl chains in the membrane from 18:1 to 16:1 in the membrane which helps stabilize the membrane from increased fluidity. In addition, cells can increase the production of chaperone and heat shock proteins to help refold denatured proteins and prevent aggregation. These cellular defenses all require significant amounts of energy, usually from sources like glucose, glycogen, trehalose, NADH and FADH₂. The consumption of trehalose to produce ATP may be particularly significant as trehalose helps to preserve membrane integrity, protein stability and suppress protein aggregation.

Since in some embodiments, the increase number of carbon-fixing and proton-pump proteins may increase the supply of the ATP as an energy source which may help facilitate the cellular defenses against ethanol stresses. In other embodiments, the genetically modified organism or cell line may preserve the accumulation of glucose, glycogen, trehalose, NADH and FADH₂; enhance the remodeling of the membrane, upregulate fatty acid elongation factor Elo1 and increase the proportion of acyl chains from 18:1 to 16:1 within the cellular membrane.

For a cell to counteract the stresses of ethanol, many of the cell's defense mechanisms require energy input. In some embodiments, the genetically modified organism or cell line may increase of ATP production may decrease the toxic effects of ethanol stress; decrease protein aggregation; decrease glucose, glycogen, trehalose, NADH and FADH₂ consumption; and decrease the loss of the proton gradient.

In other embodiments, the genetically modified organism or cell line may decrease the glycolytic negative feedback loop of an organism or cell line. Typically, during cellular respiration, high ATP levels in the cell will help prevent further ATP from being produced. If there is too much free ATP in a cell, the excess ATP will bind to phosphofructokinase, an enzyme used in glycolysis which produces ATP, and will prevent further ATP production. Since the genetically modified organism or cell line have an independent source to create the proton gradient, the typical glycolytic negative feedback loop may be bypassed.

In some embodiments, certain genes for growth of hair, feather, scale or other similar structures may be deactivated so that the organism has the maximal amount of surface area to absorb light. Also, other genes that are used for regulatory process that may limit the carbon-fixing or proton-pump protein functions of the genetically modified organism or cell line may also be deactivated.

Some organisms are able to live in symbiotic relationships with other organisms. Often times these relationships will provide mutual benefits to one another sometimes in the form of protection or in food sources. The present invention may utilize these relationships to increase the production of energy sources, such as carbohydrates or ATP, or the desired organic compound. In some embodiments, the genetically modified organism or cell line may further comprise utilization of an additional organism or cell line. In particular embodiments, the additional organism or cell line may also be genetically modified. In some embodiments, the genetically modified organism or cell line and the additional organism or cell line are symbiotic.

As a result of the various possible effects of integrating carbon-fixing and proton-pump proteins into the cell, in particular embodiments, the genetically modified organism or cell line may provide for a higher quality and a higher activity of organic compounds, including biologics and biofuels.

In some embodiments, the genetically modified organism or cell line may also use photocatalysts. In some embodiments, photocatalysts may be used to help generate free radicals and may produce hydrogen fuel. In other embodiments, hydrogen powered cells may be produced. In particular embodiments, the genetically modified organism or cell line may also further include metal catalysts. These metal catalysts may include, but are not limited to, chromium, copper, iron, nickel, platinum, palladium, titanium dioxide or zirconium dioxide. The protons that are pumped by the proton-pump proteins can then interact and bind to the free electrons at the metal sites that will be enclosed in a matrix of metal oxide. The metal oxide can be formed by titanium dioxide and zirconium dioxide. In some embodiments, hydrogen fuel may be produced through the invention.

In other embodiments, the invention may be further coupled with molecular machines. Molecular machines may include cellular components to accomplish various tasks in the cell. Examples of molecular machines include mechanical components such as joints, valves, gears, propellers, ratchets and others to form machines that can act as motors, tweezers, vehicles, assembly lines, controlled release systems, switches, transportation networks, among others. In particular embodiments, the invention may be used in conjunction with the F₀F₁-ATP Synthase motor to produce ATP.

In other embodiments, the invention may also include use of a light-gated ion channel. Light-gated ion channels are pores that can transport materials through the pore in response to light. In particular embodiments, light-gated ion channels, such as channelrhodopsin or nicotinic acetylcholine receptor, may be used to produce electrical signals from light absorption. In particular embodiments, such electrical signaling may be utilized in computers, cars, airplanes, buildings, and any other non-organic application that utilizes this form of electrical signaling.

In certain embodiments, the invention may be used by placing the proton-pump protein into a capsule-like enclosure which and may be able to produce hydrogen gas.

In certain embodiments, the genetically modified organism or cell line may include creating a facility equipped maximize light exposure and absorption of the proton-pump protein and its energy production. In particular embodiments, facility may include maximizing absorption of the proton-pump protein by providing lighting that may be adjusted to optimize absorption for the selected proton-pump protein and genetically modified organism.

In some embodiments, a composition may be prepared to include a genetically modified organism or cell line according to any of the disclosure above. In other embodiments, the composition may include an organism or cell line genetically engineered with an added gene for at least one carbon-fixing and proton-pump protein, the organism or cell line having an increased yield of a desired organic compound.

In still other embodiments, a composition an organism or cell line genetically engineered with an added gene for at least one carbon-fixing protein and at least proton-pump protein with a membrane targeting sequence, the organism or cell line having an increased yield of a desired organic compound. In particular embodiments, the composition may include an organism or cell line genetically engineered with an added gene for at least one carbon-fixing protein and at least one proton-pump protein with a mitochondrial targeting sequence, the organism or cell line having an increased yield of a desired organic compound as compared to non-genetically modified organisms or cell lines of the same type.

The invention also includes disclosure for a method of producing and harvesting at least one organic compound from an organism or cell line genetically engineered with an added gene for at least one carbon-fixing protein and at least one proton-pump protein. In some embodiments, the method may include producing and harvesting an organic compound from an organism or cell line genetically engineered with an added gene for at least one carbon-fixing protein and at least one proton-pump protein with a mitochondrial targeting sequence.

In other embodiments, the method may include increasing production of an organic compound through genetically engineering an organism or cell line to add a gene for at least one carbon-fixing protein and at least one proton-pump protein; growing the genetically modified organism or cell line; and harvesting a desired organic compound.

In particular embodiments, the method of increasing production of an organic compound may include genetically engineering a gene for at least one carbon-fixing protein to include a mitochondrial targeting sequence; genetically engineering a gene of at least one proton-pump protein to include a mitochondrial targeting sequence; genetically engineering an organism or cell line to add the gene for the genetically engineered carbon-fixing protein and proton-pump protein with the mitochondrial targeting sequence; growing the genetically modified organism or cell line; and harvesting a desired organic compound.

In some embodiments, the method of increasing production of an organic compound may include transforming an organism to add a gene for at least one carbon-fixing protein and a gene for at least one proton-pump protein; and harvesting a desired organic compound. In other embodiments, the method of increasing production of an organic compound may include genetically engineering a gene for at least one carbon-fixing protein with a membrane targeting sequence; genetically engineering a gene for at least one proton-pump protein with a membrane targeting sequence; transforming an organism to add the genetically engineered carbon-fixing protein and proton-pump protein gene; and harvesting a desired organic compound.

In other embodiments, the method of increasing production of an organic compound may include transducing a cell line to add a gene for at least one carbon-fixing protein and at least one proton-pump protein; and harvesting a desired organic compound.

In particular embodiments, the method of increasing production of an organic compound may include genetically engineering a gene for at least one carbon-fixing protein with a membrane targeting sequence; genetically engineering a gene for at least one proton-pump protein with a membrane targeting sequence; transducing a cell line to add a gene for the modified proton-pump protein; and harvesting a desired organic compound.

In some embodiments, the method of production for an organic compound may include genetically engineering an organism or cell line to add a gene for at least one carbon-fixing protein and at least one proton-pump protein; and increasing the yield of a desired organic compound.

In still other embodiments, the method of production for an organic compound may include transforming an organism to add a gene for at least one carbon-fixing protein; transforming an organism to add a gene for at least one proton-pump protein; and increasing the yield of a desired organic compound.

In particular embodiments, the method of production for an organic compound may include transducing an cell line to add a gene for at least one carbon-fixing protein; transducing an cell line to add a gene for at least one proton-pump protein; and increasing the yield of a desired organic compound.

In some embodiments, the method of production for an organic compound may include genetically engineering a gene for at least one carbon-fixing protein with a membrane targeting sequence; genetically engineering a gene for at least one proton-pump protein with a membrane targeting sequence; genetically engineering an organism or cell line to add the gene for at least one carbon-fixing protein and proton-pump protein; and increasing the yield of a desired organic compound.

In other embodiments, the method of production for an organic compound may include genetically engineering a gene for at least one carbon-fixing protein with a mitochondrial targeting sequence; genetically engineering a gene for at least one proton-pump protein with a mitochondrial targeting sequence; genetically engineering an organism or cell line to add the gene for at least one carbon-fixing and at least one proton-pump protein; and increasing the yield of a desired organic compound.

In some embodiments, the methods may include the organic compound being a biologic or a biofuel. In other embodiments, the method may increase the yield of the desired organic compound is compared to non-genetically modified organisms or cell lines of the same type.

The disclosed genetically modified organisms and cell lines; methods and compositions may have a wide variety of uses. For example, in some embodiments, genetically modified organisms and cell lines may be used in recycling for environmental efforts. Wastewaters, which include human, animal and plant wastes, along with industrial emissions of carbon dioxide, may provide an ample supply of nutrients for the genetically modified organisms and cell lines. Likewise, after harvesting the desired organic compound from the genetically modified organisms or cell lines, the leftover product may be used as animal feedstock or fertilizer. This system may help to clean and reutilize polluted environments while also producing desired organic compounds, such as biologics or biofuels. Thus, the present disclosure provides for production of organic compounds as well as conservation and utilization of alternative carbon sources.

The following examples are set forth to assist in understanding the invention and should not be construed as specifically limiting the invention described and claimed herein. Such variations of the invention, including the substitution of all equivalents now known or later developed, which would be within the purview of those skilled in the art, and changes in formulation or minor changes in experimental design, are to be considered to fall within the scope of the invention incorporated herein.

### EXAMPLES

### Example 1 (Prophetic)

### Genetically Engineering Escherichia coli with RuBisCo and Proteorhodopsin

Escherichia coli (E. coli) is selected to be genetically engineered with a carbon-fixing protein, namely RuBisCO, and a proton-pump protein, namely proteorhodopsin. Proteorhodopsin (PR) is a homolog of bacteriorhodpsin which functions as a light-driven, proton-pump protein. PR expresses well in E. coli as the preferred proton-pump protein for this system. Homologous recombination is selected to transform the E. coli.

Genomic DNA is isolated from an organism which contains a native gene for RuBisCO and PR. The RuBisCO and PR genes may be isolated through restriction enzyme and gel electrophoresis techniques known to those of skill in the art. Amplification of the isolated RuBisCO and PR genes is performed through PCR reactions optimize to produce the highest yield and quality available for the RuBisCO and PR gene. Primers for the PCR reaction may be designed include target gene sequences for homologous recombination with the targeted gene. In addition, primers may also be designed to add sequences for membrane targeting, and specifically, to the inner membrane of E. coli, where the ATPase synthase is located or any other additional desired sequence. After amplification, the PCR product is purified with techniques such as gel purification and precipitation

Also, β-carotene production genes may be inserted into the E. coli genome through homologous recombination or other techniques. E. coli do not naturally produce retinal (which is necessary for PR functioning). Thus, genes for crtE, crtYB, crtI and Bcmo1 or homologs thereof may be added to the E. coli to produce β-carotene which may be converted to retinal via the Bcmo1 enzyme. Alternatively, the transformed E. coli may be grown with a retinal supplemented media. Likewise, other desired gene additions, like additional proton-pump protein, fatty acid synthesis or carbon-fixing genes may be transformed into E. coli using the same or similar techniques.

An appropriate plasmid construct is chosen to include a desired selectable marker, such as drug resistance or color marker. The RuBisCO, PR or modified RuBisCo or PR PCR product is inserted into the plasmid construct. The E. coli is grown to an appropriate concentration for the desired transformation technique.

The plasmid is introduced to the E. coli through techniques such as electroporation. After electroporation, the E. coli is plated to incubate at least overnight. If using a selection technique, such as drug resistance, the electroporated cells are plated with the appropriate selection compound. After incubating overnight, test colonies growing on the selection plate are further cultured. PCR and DNA sequencing is used to confirm insertion of the RuBisCo, PR or modified RuBisCo or PR gene in the test E. coli colonies. SDS-PAGE and Western blotting using antibodies raised against the RuBisCo and PR protein will be used to confirm the expression of the PR protein. Additional carbon-fixing genes or other genes to enhance the proton-pump protein, carbon-fixing protein or other enhancing proteins may be included.

### Example 2 (Prophetic)

### Genetically Engineering Yeast with RuBisCO and Bacteriorhodopsin

Yeast strains, such as Saccharomyces cerevisiae or Schizosaccharomyces pombe, may be transformed through homologous recombination with RuBisCO and bacteriorhodopsin (BR) through the techniques described in Example 1. In the selected yeast strain, additional sequences (such as the membrane targeting sequences or mitochondrial targeting sequence) as well as other desirable genes may also be transformed into the yeast genome. Like E. coli, some yeast strains do not produce β-carotene or retinal. Thus, the appropriate genes (i.e., crtE, crtYB, crtI and Bcmo1 or homologs thereof) should be genetically engineered into the yeast genome or the appropriate supplemental retinal should be added in the same manner as the BR gene. Additional carbon-fixing genes or other genes to enhance the proton-pump protein, carbon-fixing protein or other enhancing proteins may be included.

### Example 3 (Prophetic)

### Genetically Engineering Mammalian Cell Lines with RuBisCO and Bacteriorhodopsin

Cell lines, such as A549 or HEK293, are selected to be transformed with RuBisCO and BR through transduction with lentivirus. The selected cell lines are prepared to the appropriate concentration. Similarly to Example 1, the RuBisCO and BR genes are isolated and amplified from isolated genomic DNA. The RuBisCO and BR genes, selection markers and any additionally desired genes or sequences are inserted into a transfer vector with long terminal repeats (LTRs) and the Psi-sequence of HIV-1. Additional desired genes may also be included in the transfer vector. The desired infection system is created with the selected transfer vector plasmid, packaging plasmid and heterologous envelop vector plasmid best optimized for the cell line to create viral particles.

The lentiviral virus is added to the selected cell line to infect the cells. Positively infected cells carrying the RuBisCO and BR genes are selected for by incubating with media with the appropriate selection compound. After incubating, test cells lines in the selection method and continue culture of the positively selected colonies. The infection event produces a mixed population of cells expressing the RuBisCO and BR genes at different levels based on where the viral genome has integrated in the host genome. To optimize expression, single cell clones are individually sorted and expanded. The single cell clones are then tested for expression and classified by their expression level of the desired gene such as RuBisCO or BR. PCR and DNA sequencing is used to confirm insertion and proper sequence of the RuBisCO, BR or modified RuBisCO or BR gene into the transduced cells.

In the foregoing description, numerous details are set forth. It will be apparent, however, that the disclosure may be practiced without these specific details. Whereas many alterations and modifications of the disclosure will no doubt become apparent to a person of ordinary skill in the art after having read the foregoing description, it is to be understood that any particular embodiment shown and described by way of illustration is in no way intended to be considered limiting. Therefore, references to details of various embodiments are not intended to limit the scope of the claims, which in themselves recite only those features regarded as the disclosure.

1. A genetically modified organism or cell line comprising:
an organism or cell line genetically engineered with an added gene for at least one carbon-fixing protein and at least one proton-pump protein.

2. The method of claim 1, wherein the at least one proton-pump protein is a light-driven proton-pump protein.

3. The method of claim 1, wherein the at least one proton-pump protein is an archaerhodopsin, bacteriorhodopsin, opsin, proteorhodopsin, rhodopsin, xanthorhodopsin, homologs thereof or combinations thereof.

4. The method of claim 3, wherein the at least one proton-pump protein absorbs light between about 100 nm and about 1 μm.

5. The method of claim 4, wherein the at least one proton-pump protein absorbs light between about 300 nm and about 750 nm.

6. The method of claim 5, wherein the at least one proton-pump protein absorbs light between about 450 nm and about 650 nm.

7. The method of claim 6, wherein the at least one proton-pump protein absorbs light between about 450 nm and about 550 nm.

8. The method of claim 6, wherein the at least one proton-pump protein absorbs light between about 550 nm and about 600 nm.

9. The method of claim 1, wherein the at least one organic compound is a biologic or a biofuel

10. The method of claim 9, wherein the biologic is selected from the group consisting of allergenics, antibodies, blood products or derivative thereof, enzymes, growth factors, hormones, immunomodulators, interferons, interleukins, polypeptides, proteins, serum, tissues, toxins and vaccines.

11. The method of claim 10, wherein the antibody is selected from the group consisting of an antitoxin, IgA, IgD, IgE, IgG, IgM and combinations thereof.

12. The method of claim 11, wherein the antibody is a monoclonal, polyclonal or bispecific antibody.

13. The method of claim 10, wherein the blood product is selected from the group consisting of red blood cells, blood plasma, white blood cells, platelets, derivatives thereof and combinations thereof.

14. The method of claim 10, wherein the enzyme is selected from the group consisting of amidases, amylases, catalases, cellulases, dehydrogenases, endonucleases, hemicellulases, hydrolases, isomerases, kinases, ligases, lipases, lyases, lysozymes, pectinases, peroxidases, phosphateses, polymerases, proteases, oxidases, oxidoreductases, reductases, transferases and combination thereof.

15. The method of claim 10, wherein the hormone is selected from the group consisting of adiponectin, adrenocorticotropic hormone, androgen, angiotensinogen, antidiuretic hormone, amylin, atrial-natriuretic peptide, brain natriuretic peptide, cacitonin, cholecystokinin, cortisol, corticotrophin-releasing hormone, cortistatin, enkephalin, endothelin, epinephrine, estrogen, erythropoietin, follicle-stimulating hormone, galanin, gastric inhibitory polypeptide, gastrin, ghrelin, glucagon, glucagon-like peptide-1, glucocorticoid, gonadotropin-releasing hormone, growth hormone, growth hormone-releasing hormone, hepcidin, human chorionic gonadotropin, human placental lactogen, humoral factors, inhibin, insulin, insulin-like growth factor, leptin, leukotriene, lipotropin, luteinizing hormone, melatonin, melanocyte stimulating hormone, mineralocorticoid, motilin, orexin, oxytocin, pancreatic polypeptide, parathyroid, pituitary adenlate cyclase-activating peptide, progesterone, prolactin, prolactin releasing hormone, prostacyclin, prostaglandins, relaxin, renin, secosteroid, secretin, somatostatin, testosterone, thrombopoietin, thromboxane, thyroid-stimulating hormone (TSH), thyrotropin-releasing hormone, thyroxine, triiodothyronine, vasoactive intestinal peptide and combinations thereof.

16. The method of claim 10, wherein the interferon is selected from the group consisting of interferon type I, interferon type II, interferon type III and combinations thereof.

17. The method of claim 10, wherein the interleukins is selected from the group consisting of interleukin-1, interleukin-2, interleukin-3, interleukin-4, interleukin-5, interleukin-6, interleukin-7, interleukin-8, interleukin-9, interleukin-10, interleukin-11, interleukin-12, interleukin-13, interleukin-14, interleukin-15, interleukin-16, interleukin-17, interleukin-18, interleukin-19, interleukin-20, interleukin-21, interleukin-22, interleukin-23, interleukin-24, interleukin-25, interleukin-26, interleukin-27, interleukin-28, interleukin-29, interleukin-30, interleukin-31, interleukin-32, interleukin-33, interleukin-34, interleukin-35, interleukin-36, interleukin-37 and combinations thereof.

18. The method of claim 10, wherein the vaccine is selected from the group consisting of whole-cell vaccine, DNA vaccine, RNA vaccine, protein-based vaccine, peptide-based vaccine, attenuated organism vaccine, attenuated virus and combinations thereof.

19. The method of claim 18, wherein the vaccine provides immunity from a disease selected from the group consisting of acquired immune deficiency syndrome, African swine fever, anthrax, bubonic plague, cervical cancer, chicken pox, Coxsackie, dengue fever, diphtheria, Ebola virus, echovirus, encephalitis, gastroenteritis, hepatitis, herpes, human immunodeficiency disease (HIV-1 or HIV-2), influenza, lower respiratory tract infection, Lyme disease, Marburg, measles, monkeypox, mumps, Norwalk virus infection, papillomavirus, parainfluenza, parvovirus, pertussis, picorna virus infection, pneumonia, pneumonic plague, polio, rabies, rotavirus infection, rubella, shingles, smallpox, swine flu, tetanus, tuberculosis, typhoids and yellow fever.

20. The method of claim 10, wherein the biologic is used to treat a condition selected from the group consisting of ankylosing spondylitis, autoimmune diseases, cancer, Crohn's disease, diabetes, gout, indeterminate colitis, inflammatory bowel disease, psoriasis, psoriatic arthritis, rheumatoid arthritis, ulcerative colitis, uveitis and viral infection.

21-129. (canceled)
